(19) United States
(12) Patent Application Publication
(10) Pub. No.: US 2005/0049273 A1
(43) Pub. Date: Mar. 3, 2005

(54) Acridone derivatives as anti-herpesvirus agents

(76) Inventors: Kenneth F. Bastow, Chapel Hill, NC (US); Christopher T. Lowden, Raleigh, NC (US)

Correspondence Address: MYERS BIGEL SIBLEY & SAJOVEC, PO BOX 37428, RALEIGH, NC, 27627, US

(21) Appl. No.: 10/651,876

(22) Filed: Aug. 29, 2003

(51) Int. Cl. A61K 31/473; A61K 31/382; A61K 31/353; A61K 31/12

(52) U.S. Cl. 514/297; 514/437; 514/455; 514/680

(57) ABSTRACT

Methods of treating a herpes virus infections are described. The methods involve administering to a subject in need thereof a compound of Formula I or Formula II below: 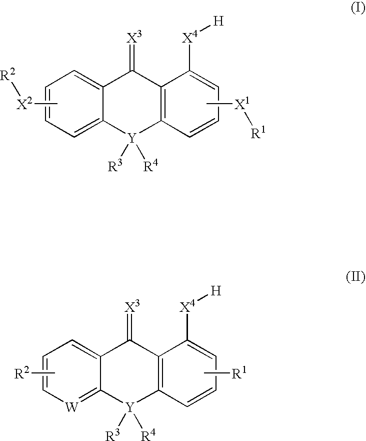
or a pharmaceutically acceptable salt thereof in an amount effective to treat the infection. In such compounds, W is N or CR⁵; X³ and X⁴ are each independently O or S; and Y is N, O, S or C.

### FIELD OF THE INVENTION

The present invention concerns compounds useful for the treatment of viral infections, particularly for the treatment of herpes simplex virus and cytomegalovirus infections.

### BACKGROUND OF THE INVENTION

Several types of naturally occurring and synthetic derivatives of 10H-acridin-9-one (acridone) are known as investigational antiviral agents and they are of medicinal interest as a group due to their diverse and atypical mechanisms of action (structures are shown in FIG. 1). 10-Carboxymethyl-acridone (10-CMA) is a potent low molecular weight inducer of interferon but may also have other mechanisms of action. For example, adenovirus type 6 is inhibited directly in vitro by 10-CMA (V. Zarubaev et al., Antiviral Res. 58, 131-137 (April 2003). The replication of human immunodeficiency virus (HIV) in human peripheral blood mononuclear cells is also inhibited by 10-CMA but with only marginal selectivity (I. Taraporewala et al., J Med. Chem., 35, 2744-2752 (1992)). In the same study, derivatives of dercetin, a sponge metabolite, inhibited HIV-1 replication in MT-4 lympocytes with greater than 16-fold selectivity. The dercetin-type of antiviral compound was proposed to inhibit HIV-1 binding to cells as well as exert other actions possibly linked to an interaction with the HIV-1 DNA replication intermediate. Of the 1-hydroxy acridone sub-class, Citrusinine-I, Citpressine-I and related phytochemicals are inhibitors of Herpes Simplex Virus (HSV) and Human Cytomegalovirus (HCMV) replication in cell culture with apparent selectivity ranging from two- to ten-fold. These agents likely target the viral-encoded enzyme ribonucleoside diphosphate reductase and thereby deplete the host of deoxyribonuclotides used to sustain efficient viral DNA replication (N. Yamamoto et al., Antiviral Res., 12, 21-36 (1989)). Another constituent of Citrus plants, 5-hydroxynoracronicine, blocks the activation of Epstein-Barr Virus (EBV) early antigen at submicromolar concentration but neither the selectivity nor a potential mechanism was elaborated (Y. Takemura et al., Planta. Med. 61, 366-367 (1995)). The synthetic 1-hydroxy acridones with antiviral activity include several 1,3-dihydroxyacridone derivatives, which inhibit HSV replication in Vero cells with modest (two- to five-fold) selectivity (K. Bastow et al., Biorg. Med. Chem., 2, 1402-1411 (1994)). Two cellular enzymes, protein kinase C (PKC) sub-type δ and DNA topoisomerase II, were proposed as potential drug targets of those analogs but the latter was excluded later primarily on the basis of structure activity information (P. Akanitapichat et al., Antiviral Res. 45, 123-134 (2000)). In the same study, 5-chloro-1,3-dihydroxyacridone (1) was discovered and designated as the lead compound because of higher selectivity (26-fold) of action. Subsequent definition of the antiviral blockade induced by the lead suggested that an undefined defect in viral (B-type) capsid competency precluded normal HSV DNA packaging in 1-treated cells (P. Akanitapichat and K. Bastow, Antiviral Res. 53, 113-126 (2002)). Another synthetic series exemplified by RD6-5071 was recently reported to inhibit chronic HIV-1 infection of various myeloid cell lines. The selectivity of RD6-5071 is about ten-fold and the antiviral mechanism occurs in part at the viral transcription level; interestingly, inhibition of cellular PCK was also considered as a possible drug target (M. Fujiwara et al., Antiviral Res., 43, 189-199 (1999)).

### SUMMARY OF THE INVENTION

A method of treating a herpes virus infection, particularly a beta-herpes virus infection, in a subject in need thereof, comprising administering to said subject a compound of Formula I: 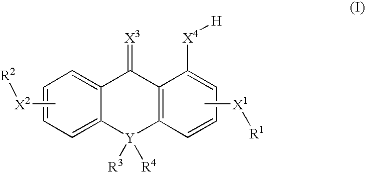
or a pharmaceutically acceptable salt thereof in an amount effective to treat said infection, wherein:

R¹ and R² are each independently selected from the group consisting of H and alkyl;

X¹, X², X³ and X⁴ are each independently selected from the group consisting of O and S;

Y is selected from the group consisting of N, O, S and C;

R³ is selected from the group consisting of H and alkyl, subject to the proviso that R³ is absent when Y is O or S; and

R⁴ is selected from the group consisting of H and alkyl, subject to the proviso that R⁴ is absent when Y is O, S or N.

A further aspect of the present invention is a method of treating a hperes virus infection, particularly an alpha-herpes virus infection, in a subject in need thereof, comprising administering to the subject a compound of Formula II: 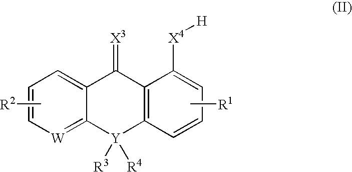
or a pharmaceutically acceptable salt thereof in an amount effective to treat said infection, wherein:

W is selected from the group consisting of N and CR⁵;

R¹, R² and R⁵ are each independently selected from the group consisting of H, alkyl, hydroxy, alkoxy and halo;

X³ and X⁴ are each independently selected from the group consisting of O and S;

Y is selected from the group consisting of N, O, S and C;

R³ is selected from the group consisting of H and alkyl, subject to the proviso that R³ is absent when Y is O or S; and

R⁴ is selected from the group consisting of H and alkyl, subject to the proviso that R⁴ is absent when Y is O, S or N.

A further aspect of the present invention is a compound of Formulas I or II as described above, or a pharmaceutically acceptable salt thereof, which are useful in the methods described herein and for the preparation of medicaments as described herein.

A further is a pharmaceutical formulation comprising a compound of Formula I or II as described above in a pharmaceutically acceptable carrier.

A Still further aspect of the present invention is the use of a compound of Formula I or Formula II as described herein for the preparation of a medicament for carrying out a method of treatment as described herein.

The foregoing and other objects and aspects of the present invention are explained in greater detail in the drawings herein and the specification set forth below.

### BRIEF DESCRIPTION OF THE DRAWINGS

FIG. 1. Acridone derivatives with antiviral activity.

FIG. 2. Inhibition of HCMV plaque formation and cytopathogenicity by 3,7-dihydroxy-1-hydroxyacridone (2): HEL Cells infected with a low multiplicity of HCMV were treated with various concentrations of compound 2 and plaques were counted after 10 days of continuous treatment. Details of the HCMV plaque-elimination assay are given in the examples below. The graphed data shows the dose-dependent antiviral activity of 2, with values representing the mean and standard deviation of triplicate treatments from two independent experiments (panel A).

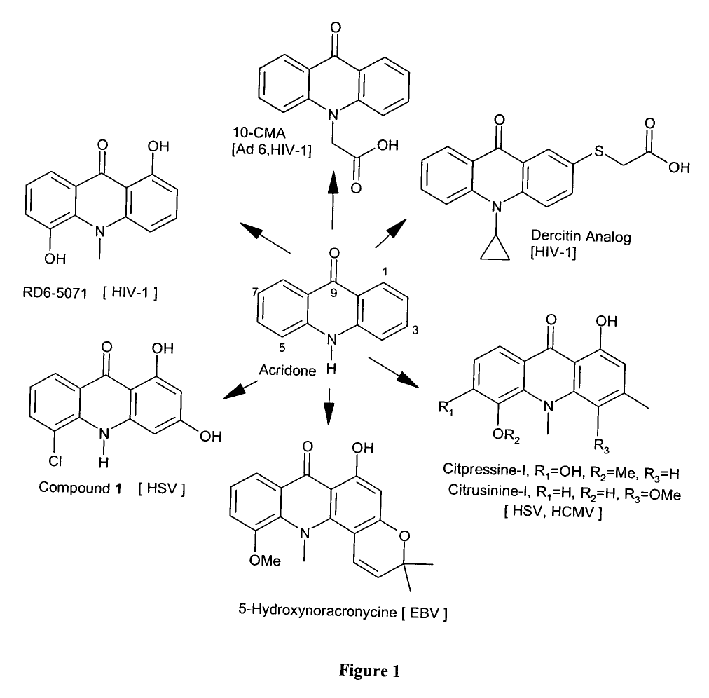

HEL Cells were replicated in the presence of either 0.5 or five micromolar compound 2 for 12 days (four passages) then they were compared to untreated HEL cells as a host for HCMV replication in the presence or absence of the drug. Treatment had no effect on cell morphology or cell viability as reflected by microscopic appearance and by replication rate respectively. Cultures were infected with ten-times the viral load used for plaque assay and were fixed, stained and photographed after seven days of infection (panel B). A detailed method is described in the examples below. The horizontal rows labeled as HEL, HEL (0.5) and HEL (5.0) represent cells cultured in the absence or presence of 0.5 and 5 micromolar 2 respectively. Labeled columns indicate mock-infected (M), infected (I) and treated after viral infection with the same two concentrations of the compound.

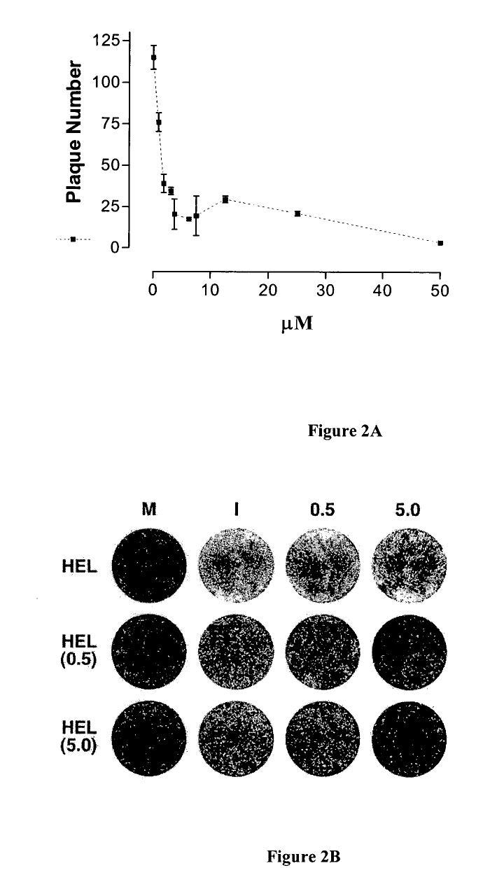

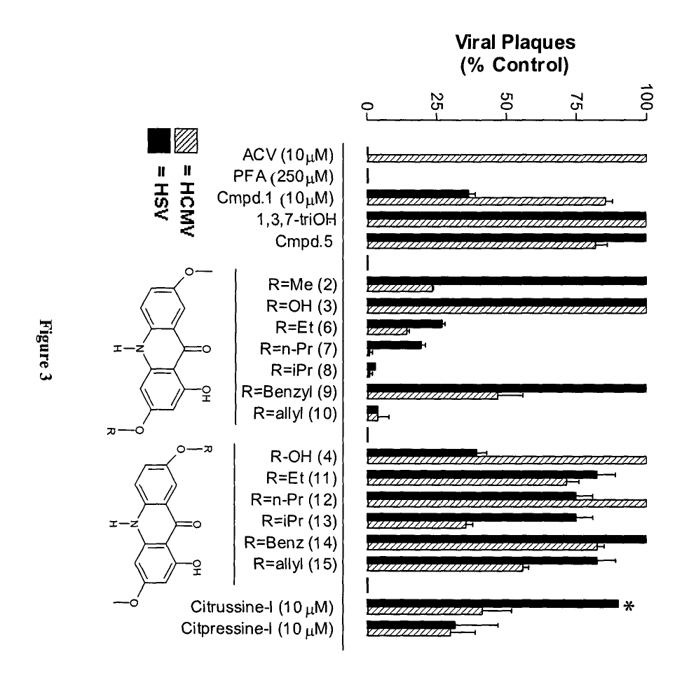

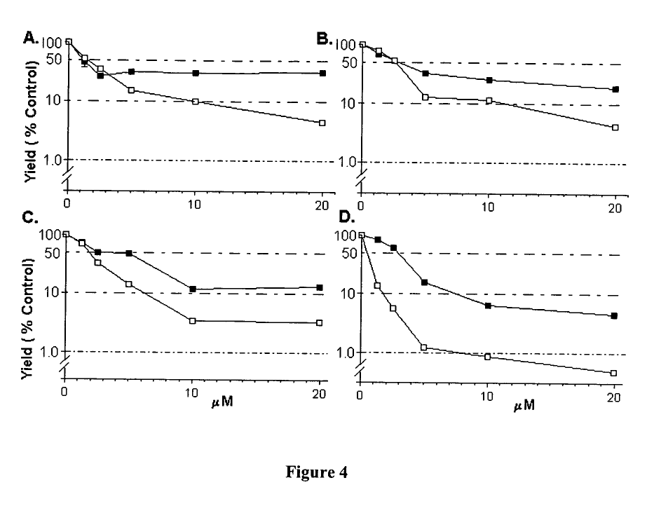

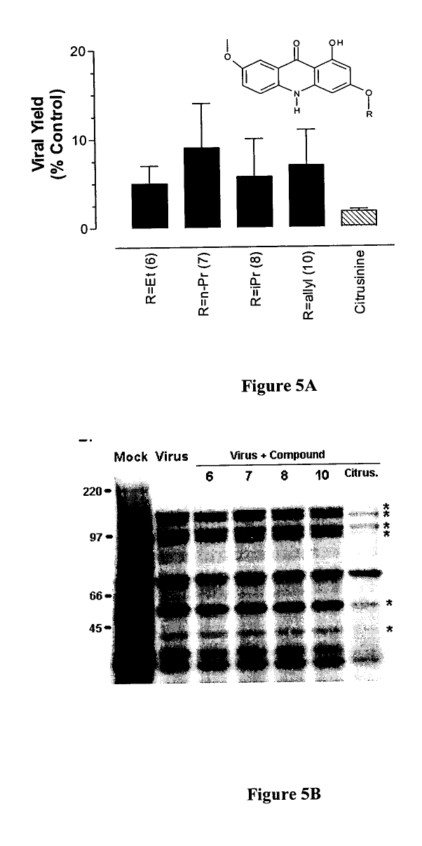

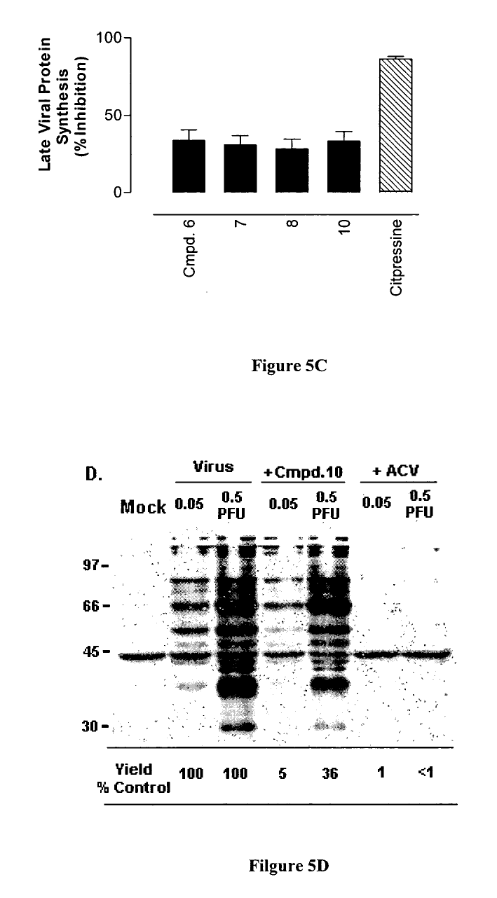

FIG. 3. Comparison between bis-alkoxylated 1-hydroxyacridones as HSV-2 and HCMV inhibitors: A plaque-elimination assay was used to screen compounds as inhibitors of HSV-2 and HCMV in Vero and HEL cells respectively (see examples). Compounds were tested at five micromolar concentration except for 5-chloro-1,3-dihydroxyacridone (1), 1-hydroxy-3,7-dimethoxyacridone (2) and acyclovir (ACV) which were tested at ten micromolar and for phosphonoformic acid (PFA), which was evaluated at 250 micromolar. Filled bars and hatched bars are activity against HSV-2 and HCMV respectively. The values represent mean and standard error of results from two independent experiments conducted several months apart. For control treatments (1, 2, PFA, ACV and 1,3,7-trihydroxyacridone), results were compatible with work reported herein (FIG. 2A, Table 1) and elsewhere (Bastow et al., Antimicrob. Agents, and Chemother. 23: 914-917 (1983); P. Akanitapichat et al., Antiviral Res. 45, 123-134 (2000)). The asterisk denotes that HSV plaque-size was uniformly smaller in the presence of Citrusinine I but the number of visible plaques was not reduced.

FIG. 4. Effect of viral load and serum concentration on the anti-HSV activity of 3-allyloxy-1-hydroxy-7-methoxyacridone (10). Vero cells were infected with HSV-1 at either 1.0 or 0.01 PFU per cell. One hour after infection, either standard growth medium or medium supplemented with 10-fold lower serum (0.5%v/v) and various concentrations of compound 10 was added. The production of cell-associated and Teleased virus at 23 hours post-infection was measured by serial dilution and plaque assay as described in the examples. The open squares represent the condition of low multiplicity (0.01 PFU), infection. Panels A and B show the amount of cell-associated virus produced in medium containing 5% and 0.5% (v/v) serum respectively. Panels C and D show the virus released into medium supplemented with 5% and 0.5% (v/v) serum respectively. The yield of virus is plotted on a logarithmic scale to more clearly illustrate the differences apparent between treatments.

FIG. 5. Effects on HSV protein synthesis and accumulation: Vero cells were infected with HSV-2 at a multiplicity of 0.1 PFU and treated for 22 hours with compounds at twenty micromolar in medium containing 2% (v/v) serum. Cell-associated and released virus was harvested and virus production was quantified by dilution using a plaque-assay. Parallel cultures were pulse-labeled at 17 hours post-infection and total cell extracts were subsequently analyzed for late viral protein synthesis. Detailed methods are covered in the examples. The relative amount of progeny virus recovered from drug-treated cultures is shown in panels A (the apparent variation in the activity of 6-8 and 10 reflects the significant differential between inhibition of cell-associated verus released virus under the treatment condition used). The phosphorimage in panel B shows the radio-labeled proteins detected, with treatment condition indicated above each lane. Molecular mass of marker proteins in kDa is indicated in the left margin. Viral proteins denoted with an asterisk in the right margin were used for quantitative analysis of viral protein synthesis and the results obtained are represented graphically in panel C. The image in panel D is a Western immunoblot of infected cell extracts stained with a polyvalent HSV antibody (see Examples). Cells were infected with HSV-1 and treated for 16 hours either with compound 10 or ACV at 10 micromolar in medium containing 2% (v/v)serum. Additional details of specific treatments are given above each lane. The relative amount of progeny virus recovered from the medium of parallel cultures is shown under the lanes. Molecular mass of proteins is indicated in the left margin. A cross-reacting cellular protein detected in samples is possibly actin on the basis of high abundance and apparent molecular mass.

### DETAILED DESCRIPTION OF THE PREFERRED EMBODIMENTS

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like.

The term “alkoxy,” as used herein, refers to an alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like.

The term “hydroxy,” as used herein, refers to an —OH group.

The term “halo” or “halogen,” as used herein, refers to —Cl, —Br, —I or —F.

The term “treat” as used herein refers to any type of treatment that imparts a benefit to a patient afflicted with a disease, including improvement in the condition of the patient (e.g., in one or more symptoms), delay in the progression of the disease, etc.

The term “pharmaceutically acceptable” as used herein means that the compound or composition is suitable for administration to a subject to achieve the treatments described herein, without unduly deleterious side effects in light of the severity of the disease and necessity of the treatment.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

1. Active Compounds.

The methods of the present invention include the administration of compounds of Formulas I or II, while pharmaceutical compositions of the present invention comprise compounds of Formulas I or II.

As noted above, the present invention provides compounds of Formula I: 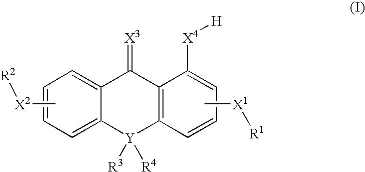
wherein:

R¹ and R² are each independently selected from the group consisting of H and alkyl (e.g., H or methyl);

X¹, X², X³ and X⁴ are each independently selected from the group consisting of O and S;

Y is selected from the group consisting of N, O, S and C;

R³ is selected from the group consisting of H and alkyl (e.g., H or methyl), subject to the proviso that R³ is absent when Y is O or S; and

R⁴ is selected from the group consisting of H and alkyl (e.g., H or methyl), subject to the proviso that R⁴ is absent when Y is O, S or N.

In an embodiment of the foregoing, X¹, X², X³ and X⁴ are all O.

In an embodiment of the foregoing, R³ and R⁴ are H or methyl.

In an embodiment of the foregoing, Y is N.

In another embodiment of the foregoing, Y is O.

In another embodiment of the foregoing, Y is S.

In another embodiment of the foregoing, Y is C.

Another embodiment of the present invention is compounds of Formula II: 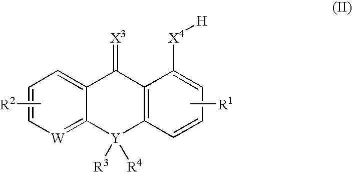
wherein:

W is selected from the group consisting of N and CR⁵;

R¹, R² and R⁵ are each independently selected from the group consisting of H, alkyl, hydroxy, alkoxy and halo;

X³ and X⁴ are each independently selected from the group consisting of O and S;

Y is selected from the group consisting of N, O, S and C;

R³is selected from the group consisting of H and alkyl, subject to the proviso that R³ is absent when Y is O or S; and

R⁴ is selected from the group consisting of H and alkyl, subject to the proviso that R⁴ is absent when Y is O, S or N and.

In an embodiment of the foregoing, W is N.

In an embodiment of the foregoing, W is CR⁵.

In an embodiment of the foregoing, R¹, R² and R⁵ are each independently selected from the group consisting of H and methyl.

In an embodiment of the foregoing, X³ and X⁴ are each O.

In an embodiment of the foregoing, Y is N.

In another embodiment of the foregoing, Y is O.

In another embodiment of the foregoing, Y is S.

In another embodiment of the foregoing, Y is C.

Compounds of Formulas I or II may be prepared by techniques such as thermal coupling as disclosed herein, or variations thereof which will be apparent to those skilled in the art given the present disclosure.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

2. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unitdose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formulas I or II, or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bistris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to compounds of formulas I or II or their salts, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

3. Methods, Dosage and Routes of Administration.

Compounds of Formulas I and II are useful in treating viral infections in human or animal subjects in need thereof. Compounds of Formula I are particularly useful for treating a beta-herpes virus infection in a human or animal subject in need thereof. Examples of such viruses and viral infections include but are not limited to herpes virus 6, herpes virus 7, and human cytomegalovirus. Compounds of Formula II are particularly useful for treating an alphaherpes infection in a human or animal subject in need thereof. Examples of such viruses and viral infections include but are not limited to herpes simplex virus, herpes virus 8, Varicella-Zoster virus and herpes virus simiae.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any one active agent, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. As a general proposition, a dosage from about 0.1 to about 50 mg/kg, or total dosage for the subject of 1 to 1000 mg, will have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. Typically, a dosage from about 0.5 mg/kg to 5 mg/kg may be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once per day for a period of two to three weeks or until the condition is essentially controlled. Lower doses given less frequently can be used prophylactically to prevent or reduce the incidence of recurrence of the infection.

The present invention is explained in greater detail in the following non-limiting Examples

### EXAMPLES

The present invention is based, among other things, on the finding that the antiviral activity spectrum of synthetic 1-hydroxyacridones may be extended to include the significant pathogen HCMV. Of this sub-class, C-3 variable bis-alkoxylated derivatives (compounds 6-8 and 10), are the most intriguing because like the Citrus alkaloids discovered by Yamamoto et al., they inhibit a productive HSV infection as well. However, on the bases of structure-activity relationships and preliminary information about mode of action, these novel dual inhibitors appear to be unique amongst antiviral acridones and therefore they are useful templates for anti-herpes drug research and development.

¹H NMR spectra were recorded on a Varian 300 MHz spectrometer with Me4Si as the internal reference. Mass spectra of compounds 2-5 were measured using an Hitachi M-80 mass spectrometer and for 6-15, by ESI-MS analysis using a PE-Sciex API-3000 LC/MS/MS with turbo spray ion source operating at ⁻4.2 KV. Elemental analysis of compounds 2-5 was performed by Atlantic Microlabs (Norcross, Ga). Purity of the parallel series and selected other compounds was monitored using HPLC. This analysis with uv (250 nm) detection in MeOH:water (80:20) used an Agilent 1100 system equipped with an Agilent 4.6 mm ID×15 cm ZORBAX Eclipse XDB -C8 column. The flow rate and run time were 1.0 mL/min., and 10 min., respectively.

### Examples 1-4

### Preparation of 1-hydroxyacridone analogs 2-5

The synthetic approach used for single analog synthesis is illustrated in Scheme 1. This thermal coupling reaction is an expedient alternative to more commonly used synthetic routes that involve refluxing in n-butanol and zinc chloride (G. Hughes and Ritchie, Aust. J Sci. Res. 423-431 (1951)) or n-heptanol and p-toluene sulfonic acid (R. Smolders et al., Bull. Soc. Chim. Belg.. 93, 239-240 (1984)) for the coupling of anthranilic acids with phloroglucinol or resorcinol derivatives. The methodology was optimized to define the structure activity relationship around compound 1 (C. Lowden, Ph.D. Thesis, UNC-Chapel Hill (2002)) and was subsequently adapted to a more diverse set of acridone targets including compounds 2-5.

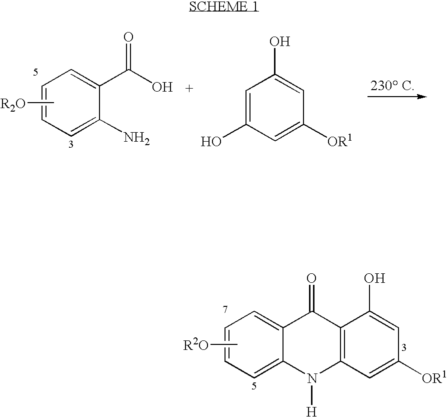

3,7-dimethoxy-1-hydroxy-acridone (2). Into a 20 mL vial was added 2-amino-5-methoxybenzoic acid (1.00 g, 6.00 mmol) and 5-methoxyresorcinol (947 mg, 6.75 mmol). The vial was sealed and heated in an oil bath at 225° C. for 35 minutes before allowing it to cool to room temperature. The resulting solids were triturated in ethyl acetate, and filtered to yield 780 mg yellow powder, 48%. NMR (D₆ DMSO) δ 3.97 (6H, s), 6.25 (1H, s), 6.47 (1H, s), 7.55-7.67 (3H, m), 11.97 (1H, s), 14.41 (1H, s); elemental analysis calculated for C₁₅H₁₃NO₄: C 66.41, H 4.83, N 5.16, Found C 66.15, H 4.92, N 5.23. HRMS m/z (rel. int. %) 271 (100) (M)⁺; calculated for C₁₅H₁₃NO₄: 271.0845, Found 271.0840.

1,3-Dihydroxy-7-methoxyacridone (3) Into a 20 mL vial was added 2-amino-5-methoxybenzoic acid (2.17 g, 6.00 mmol) and anhydrous phloroglucinol (1.64 g, 6.75 mmol). The vial was sealed and heated in an oil bath at 230° C. for 35 minutes. Upon cooling, the resulting solids were triturated in ethyl acetate and methyl alcohol before filtration. The combined filtrates were evaporated, dissolved in DMF (15 mL) and ethyl acetate (150 mL), and washed with 4-1 H₂O-saturated aqueous sodium bicarbonate (2×150 mL) followed by H₂O (2×150 mL). The organic phase was dried over sodium sulfate, filtered and the crude product was then flash chromatographed to yield 600 mg yellow powder, 18%. NMR (D₆ DMSO) δ 3.97 (3H, s), 6.10 (1H, d, J=2.4), 6.39 (1H, d, J=2.4), 7.61 (3H, m) 10.55 (1H, s), 11.84 (1H, s), 14.43 (1H, s); elemental analysis calculated for C₁₄H₁₁NO₄.1.5 H₂O: C 59.15, H 4.96, N 4.93, Found C 58.76, H 4.58, N 5.01. HRMS m/z (rel. int %) 257 (7.5) (M)⁺; calculated for C₁₄H₁₁NO₄: 257.0691, Found 257.0688; HPLC purity (retention time) 100% (2.1 min).

1,7-Dihydroxy-3-methoxyacridone (4) Into a 20 mL vial was added 2-amino-5-hydroxybenzoic acid (281 mg, 1.83 mmol) and 5-methoxyresorcinol (284 mg, 2.02 mmol). The vial was sealed and heated in an oil bath at 230° C. for 35 minutes. After cooling to room temperature, the resulting solid was then triturated in hot ethyl acetate and filtered to yield the product as a yellow solid, 335 mg, 71%. NMR (D₆ DMSO) δ 3.96 (3H, s), 6.21 (1H, d, J=2.4), 6.45 (1H, d, J=2.4), 7.41 (1H, dd, J=2.8, 7.2), 7.54 (1H, d, J=9.1), 7.61 (1H, d, J=2.8) 9.78 (1H, s), 11.97 (1H, s), 14.48 (1H, s); elemental analysis calculated for C₁₄H₁₁NO₄.0.5 H₂O: C 63.16, H 4.54, N 5.26, Found C 63.40, H 4.63, N 5.10. HRMS m/z (rel. int. %) 257 (7.5) (M)⁺; calculated for C₁₄H₁₁NO₄: 257.0688, Found 257.0693; HPLC purity (retention time) 95% (2.1 min).

3,5-Dimethoxy-1-hydroxyyacridone (5). Into a 4 mL vial was added 3-methoxy-2-aminobenzoic acid (334 mg, 2.00 mmol) and 5-methoxyresorcinol (308 mg, 2.20 mmol). The vial was sealed and heated in an oil bath at 230° C. for 35 min. The resulting solids were dissolved in ethyl acetate (100 mL) and washed with 2×100 mL 0.1OM KOH in H₂O. The organic phase was isolated, dried over sodium sulfate and absorbed onto silica gel. The material was then flash chromatographed to yield 70 mg yellow solid, 13%. NMR (D₆ DMSO) δ 3.96 (3H, s), 4.17 (3H, s), 6.28 (1H, d, J=2.0), 7.03 (1H, d, J=2.4), 7.34 (1H, t, J=8.0), 7.47 (1H, d, J=7.9), 7.87 (1H, d, J=8.3), 11.48 (1H, s), 14.35 (1H, s); elemental analysis calculated for C₁₅H₁₃NO₄.0.75H₂O: C 63.26, H 5.13, N 4.92, Found C 63.07, H 4.71, N 4.96. HRMS m/z (rel. int %) 271 (8.7) (M)⁺; calculated for C₁₅H₁₃NO₄: 271.0845, Found 271.0851.

### Examples 5-14

### Parallel Preparation of Compounds 6-15

The synthetic approach used for solution phase parallel synthesis is illustrated in Scheme 2. Into each of six 20 mL vials with stir bars was added sequentially 1,3 dihydroxy-7-methoxyacridone (3, 86 mg, 0.33 mmol), cesium carbonate (98 mg, 0.30 mmol) and 1-1 DMF-acetone (4 mL). Six alkyl halides (ethyl iodide, propyl iodide, isopropyl iodide, benzyl bromide, cyclohexyl bromide, allyl bromide, 0.30 mmol) were then added separately to vials. Reactions were at room temperature for 24 hours with stirring. Acetone and some DMF were evaporated with a nitrogen stream from a manifold apparatus while the vials rested in a warm water bath. After adding ethyl acetate (8 mL) and water (8 mL) into reaction vials, they were capped and shaken vigorously. The water was removed and re-extracted in the same manner with ethyl acetate (4 mL). Organic phases combined from both extractions were washed with water (5 mL), then isolated and filtered over a pad of sodium sulfate and silica gel. The solid phase was washed with ethyl acetate (20 mL) and the filtrates were concentrated. Resultant solids were triturated in dichloromethane and filtered to yield the products (6-10) as yellow solids in 81-99% purity as estimated by peak area using HPLC analysis. A cyclohexyl bromide reaction did not yield any product. The same procedure was carried out using 1,7-dihydroxy-3-methoxyacridone (4) as the starting acridone in parallel to yield compounds 11-15 in 69-100% purity. Again, the cyclohexyl bromide reaction in parallel was not successful.

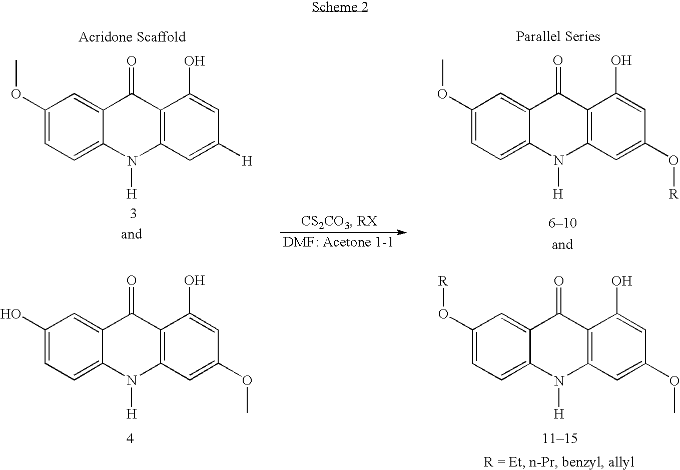

3-Ethoxy-1-hydroxy-7-methoxyacridone (6) NMR (CDCl₃) δ 1.57 (3H, t, J=7.1), 4.02 (3H, s), 4.18-4.25 (2H, m), 6.26 (1H, d, J=2.4), 6.36 (1H, d, J=2.4), 7.32 (1H, d, J=9.1), 7.40 (1H, d, J=9.1), 7.84 (1H, d, J=2.6), 8.47 (1H, s), 14.21 (1H, s). LRMS m/z (rel. int. %) (M)⁻=283.9 (100); HPLC purity (retention time) 94% (2.3 min).

1-Hydroxy-7-methoxy-3-propoxyacridone (7) NMR (CDCl₃) δ 1.81 (3H, t, J=7.1), 1.93-2.00 (2H, m), 4.02 (3H, s), 4.10 (2H, t, J=6.8), 6.27 (1H, s), 6.38 (1H, s), 7.32 (1H, d, J=8.7), 7.41 (1H, d, J=6.5), 7.84 (1H, s), 8.43 (1H, s), 14.21 (1H, s). LRMS m/z (rel. int. %) (M)⁻=298.0 (100); HPLC purity (retention time) 93% (2.4 min).

1-Hydroxy-3-isopropoxy-7-methoxyacridone (8) NMR (CDCl₃) δ 1.50 (3H, s), 1.53 (3H, s), 4.03 (3H, s), 4.73-4.78 (1H, m), 6.27 (1H, s), 6.37 (1H, s), 7.32 (1H, d, J=8.7), 7.43 (1H, d, J=7.9), 7.85 (1H, s), 8.35 (1H, s), 14.20 (1H, s). LRMS m/z (rel. int. %) (M)⁻=297.9 (100); HPLC purity (retention time) 81% (2.4 min).

3-Benzyloxy-1-hydroxy-7-methoxyacridone (9) NMR (CDCl₃) δ 4.02 (3H, s), 5.35 (2H, s), 6.62 (1H, s), 7.02-7.19 (2H, m), 7.25-7.59 (6H, m), 7.80-7.88 (1H, m), 8.01 (1H, s), 14.42 (1H, s). LRMS m/z (rel. int. %) (M)⁻=345.9 (100); HPLC purity (retention time) 99% (2.3 min).

3-Allyloxy-1-hydroxy-7-methoxyacridone (10) NMR (CDCl₃) δ 4.02 (3H, s), 4.71-4.73 (2H, m), 5.44-5.60 (2H, m), 6.12-6.25 (1H, m), 6.30 (1H, d, J=1.9), 6.39 (1H, d, J=2.2), 7.29-7.46 (2H, m), 7.85 (1H, d, J=2.8), 8.45 (1H, s), 14.40 (1H, s). LRMS m/z (rel. int. %) (M)⁻=295.9 (100); HPLC purity (retention time) 91% (2.3 min).

7-Ethoxy-1-hydroxy-3-methoxyacridone (11) NMR (CDCl₃) δ 1.59 (3H, t, J=7.1), 4.00 (3H, s), 4.23-4.31 (2H, m), 6.28 (1H, s), 6.38 (1H, d, J=2.0), 7.29-7.44 (2H, m) 7.84 (1H, s), 8.36 (1H, s), 14.24 (1H, s). LRMS m/z (rel. int. %) (M)⁻=283.9 (100); HPLC purity (retention time) 92% (2.3 min).

1-Hydroxy-3-methoxy-7-propoxyacridone (12) NMR (CDCl₃) δ 1.19 (3H, t, J=7.1), 1.95-2.02 (2H, m), 3.99 (3H, s), 4.15 (2H, t, J=7.1), 6.28 (1H, s), 6.38 (1H, d, J=2.0), 7.29-7.43 (2H, m), 7.84 (1H, d, J=2.4), 8.39 (1H, s), 14.25 (1H, s). LRMS m/z (rel. int. %) (M)⁻=298.1 (100); HPLC purity (retention time) 77% (2.3 min).

1-Hydroxy-7-isopropoxy-3-methoxyacridone (13) NMR (CDCl₃) δ 1.50 (3H, s), 1.52 (3H, s), 3.99 (3H, s), 4.78-4.82 (1H, m), 6.27 (1H, s), 6.38 (1H, s), 7.28-7.41 (2H, m), 7.87 (1H, s), 8.46 (1H, s), 14.26 (1H, s). LRMS m/z (rel. int. %) (M)⁻=298.1 (100); HPLC purity (retention time) 69% (2.3 min).

7-Benzyloxy-1-hydroxy-3-methoxyacridone (14) NMR (CDCl₃) δ 4.01 (3H, s), 5.32 (2H, s), 6.28 (1H, s), 6.40 (1H, d, J=2.4), 7.32-7.63 (6H, m), 7.98 (1H, s), 8.15 (1H, s), 8.24 (1H, s), 14.20 (1H, s). LRMS m/z (rel. int. %) (M)⁻=345.9 (100); HPLC purity (retention time) 99% (2.3 min).

7-Allyloxy-1-hydroxy-3-methoxyacridone (15) NMR (CDCl₃) δ 4.02 (3H, s), 4.77-4.81 (2H, m), 5.40-5.63 (2H, m), 6.18-6.22 (1H, m), 6.28 (1H, s), 6.37 (1H, d, J=10.0), 7.28-7.47 (2H, m), 8.01 (1H, s), 8.32 (1H, s), 14.21 (1H, s). LRMS m/z (rel. int. %) (M)⁻=295.8 (100); HPLC purity (retention time) 100% (2.3 min).

Chemistry Results. In previous work, a variety of 1-hydroxyacridone analogues including 1, 4 and 1,3,7-trihydroxy acridone were prepared by condensation of anthranilic acid and resorcinol (each appropriately substituted) in n-butyl alcohol at reflux in the presence of zinc chloride (Hughes and Richie, supra 1951, Bastow et al., supra 1994, Akanitapichat et al., supra 2000). Compound 2 was then synthesized by selective alkylation of 1,3,7-trihydroxyacridone in 18% yield (C. Lowden,. Masters's Thesis, UNC-Chapel Hill (1995)). Although the Hughes and Richie reaction is somewhat versatile, its efficiency and the ease of product purification was found to vary considerably, therefore an alternate one-step synthetic route to targets 2-5 was investigated (Scheme 1). The reaction, a simple thermal condensation, was originally developed to define the structure activity relationship around compound 1, the HSV lead (C. Lowden, Ph.D. Thesis, UNC-Chapel Hill (2002)). Thermal coupling proved to be a superior route to compounds 2 and 4 and also afforded 3, the acridone skeleton used to prepare the variable alkoxylated series at C-3 (compounds 10-15), in parallel and compound 5, the 3,5-regioisomer of the HCMV lead (2). The goal of the parallel synthesis was to develop a viable method for the rapid production of compounds closely related to 2 as an exploratory series for preliminary biological evaluation. Pilot studies were undertaken to examine the feasibility of selective O-alkylation of compounds 3 and 4. Use of the Mitsunobu reaction proved to be selective for phenolic alkylation, but removal of the byproducts in a parallel fashion was problematic. Therefore reaction with alkyl halides was explored as a potential route (Scheme 2). In order to facilitate the selective alkylation of the hydroxyl at C-3 or C-7 over the secondary amine, less than one equivalent of alkyl halide was used in a room temperature reaction. Non-reacted acridone (3 or 4) was readily removed with aqueous potassium hydroxide, probably due to the presence of an acidic phenol (the phenol in the 1-position is much less acidic due to hydrogen bonding with the carbonyl). Removal of alkyl halides was accomplished through filtration, subsequent to trituration in dichloromethane. The quantity of bis-alkylated impurities varied depending on the alkylating agent but the trituration step proved to be largely selective for the desired product. On the basis of TLC analysis, the alkylations were usually complete in the first hour; however, alkylating agents with branching on the alpha carbon reacted much slower. Alkylation with cyclohexyl bromide occurred only minimally at the 3-hydroxyl position of 3, and 4 was not alkylated using cyclohexyl bromide even after heating. Presumably, this result can be attributed to steric hindrance around the bromide leaving group. Yields were approximately 50% for the reaction, and purity ranged from 69-100% (see above). Mass spectral and HPLC analysis indicated that either starting material or bis-alkylated products were the main impurities. The structures of the ten products that were isolated from the parallel synthesis (6-15), are depicted in Scheme 2 and FIG. 3.

### Examples 15-20

### Biological Activity

Reagents and drugs. Acyclovir (ACV) and Foscamet (PFA) were obtained from Sigma Chemical Co,. (St Louis, Mo.). 5-Chloro-1,3,-dihydroxyacridone (1) and 1,3,7-trihydroxyacridone were prepared as described (Akanitapichat et. al. 2000). The original source of the Citrus alkaloids, Citrusinine-I and Citpressine-I was Dr. Hiroshi Furukawa (K. Bastow et al., Biorg. Med. Chem., 2, 1402-1411 (1994)). For biological testing, all compounds were dissolved in DMSO as 20 mM stock solutions except PFA, which was prepared at similar concentration but in sterile phosphate buffered saline. TRAN³⁵S-LABEL™ (E.coli hydrolysate labeling reagent containing 70% L-Methionine, [³⁵S]; >10,000 Ci/mmol) was purchased from ICN Radiochemicals (Irvine, Calif.). The source and use of the polyclonal rabbit antibody against HSV infected cells was described previously (P. Akanitapichat and K. Bastow, Antiviral Res. 53, 113-126 (2002)). All other chemicals were reagent grade.

Cells and virus. The African green monkey kidney (Vero 76: ATCC No.: CRL 1587) and human embryonic lung fibroblasts (HEL, ATCC No.: CCL 137) cells were purchased from the UNC Lineberger Comprehensive Cancer Center (Chapel Hill, N.C.). Cells were routinely cultured in RPMI-1640 medium supplemented with 10% (v/v) fetal calf serum and 100 μg/mL of kanamycin (designated “standard” medium) in a humidified 5% (v/v) CO₂ incubator at 37° C. Throughout the course of experiments, HEL cells were sub-cultured at 1:3 dilution and were not used beyond seven passages from receipt. A high titre stock of HSV-2 (strain 186), a generous gift of Dr. S. Bachenheimer (Microbiology, UNC-CH), was stored frozen as aliquots and used directly for the present work. The same HSV-2 strain was used for the original work on Citrus alkaloids (N. Yamamoto et al., Antiviral Res., 12, 21-36 (1989)). HCMV (Towne, VR977, Lot 6W) was purchased from the American Type Culture Collection (Rockville, Md.) and a working stock was prepared (2×10⁵ PFU per mL) by low multiplicity infection of HEL cells. The source and maintenance of the HSV-1 (KOS) strain was as described (Akanitapichat and Bastow, supra 2002).

Virus and cell growth inhibition assays. Established virus culture techniques were used (E.-S. Huang, and T Kowalnick, Diagnosis of human cytomegalovirus infection: laboratory approaches, in Molecular Aspects of Human Cytomegalovirus Diseases (Becker,Y., Daria, G., and Huang, E.-S. eds.), Springer-Verlag, Berlin, pp. 225-255 (1993); Akanitapichat et al., Antiviral Research 45: 123-134 (2000)) but with the following modifications. For the HCMV plaque-elimination assay, HEL cells were plated in standard medium at 60,000 per cm² and infected the following day with 50-100 PFU HCMV for 90 minutes with occasional agitation. The inoculate was replaced with maintenance medium containing 5% (v/v) fetal calf serum and test agents as indicated (see below). After an additional 6-10 days of culture, cells were fixed with formal saline (10% formalin in phosphate-buffered saline; PBS), stained with 0.1% (w/v) toluidine blue in PBS and plaques were scored using an inverted light microscope at 40x magnification (FIGS. 2A and 3). For ED₅₀ determination, the value was interpolated from dose-response data and is the concentration of compound that reduced plaque formation by 50% relative to control under the specified condition.

To examine whether pre-treatment of cells influenced anti-HCMV activity of 1-hydroxy-3,7-dimethoxyacridone (2), a cytopathogenicity (CPE) reduction assay was used. The protocol was like the plaque-elimination assay except HEL cells were replicated in test compound (3-12 days or from 1-4 passages) prior to infection, cultures were infected with 500-1000 PFU HCMV and after five days, cells were stained with 0.8% (w/v) crystal violet in 50% ethanol in order to achieve contrast for photography. A representative result is shown in FIG. 2B.

Three types of antiviral assay were used for HSV studies. For plaque-elimination, Vero cells (70000 per cm²) were infected with 50-100 PFU for 30 minutes with occasional agitation. The inoculate was replaced with medium containing 1% (v/v) fetal calf serum and supplemented with test agents as indicated (see below). After two days of culture, cells were processed using crystal violet staining and plaques were scored by visual examination (FIG. 3). For examination of anti-HSV activity in parallel cultures during biochemical experiments, the progeny virus obtained from cells and from the culture medium at 23 hours post-infection was determined by limiting dilution on Vero cells (FIG. 5A). Macroscopic viral plaques were scored after two days of incubation in medium containing 1% (w/v) carboxymethylcellulose and 0.5% (v/v) fetal calf serum. For ED₅₀ measurements, the concentration of compound that reduced a single-cycle of viral yield by 50% relative to the control and under the specified condition was interpolated from dose-response graphs (FIG. 4, Table 1). Over the course of ten independent experiments, the mean cell-associated viral yield was 180 PFU per cell (SD=80) and 10 PFU per cell (SD=5) for HSV-1 and HSV-2 respectively. The percent viral release (into the culture medium) was 16 (SD=7) and 0.9 (SD=0.6) for HSV-1 and HSV-2 respectively. These virologic parameters were independent of viral load and serum concentration.

TABLE 1
Comparison between inhibitors of HSV and/or HCMV replication in
cultured cells

| | Parameter (μM)^{a} | | | | |
|---|---|---|---|---|---|
| | ED₅₀ | ED₅₀ | CC₅₀ | ED₅₀ | CC₅₀ |
| Compound | HSV-1 | HSV-2 | Vero | HCMV | HEL |
| ACV | 0.2 | 0.8 ± 0.1 | >200(41) | >200(NA) | >500(430) |
| PFA | ND | 60 ± 5.0 | >500(11) | 70 ± 10 | >500(11) |
| Citrusinine-I | 30 ± 2.0 | 2.5 ± 0.7 | 27 ± 4.0 | 9.0 ± 1.5 | 41 ± 5.0 |
| 5-Chloro- | 4.3 ± 0.8 | 3.8 ± 0.3 | >50(23) | >50(25) | >50(18) |
| 1,3-di- | | | | | |
| hydroxy- | | | | | |
| acridone (1) | | | | | |
| 3,7-Di | >50(NA) | >50(NA) | >50(7) | 1.4 ± 0.3 | >50(NA) |
| methoxy-1- | | | | | |
| hydroxy- | | | | | |
| acridone (2) | | | | | |
| 3-Alloxy-1- | 2.3 ± 0.3 | 3.9 ± 1.0 | 95 ± 2.0 | >2.5(80) | >50(33) |
| hydroxy-7- | | | | | |
| methoxy- | | | | | |
| acridone (10) | | | | | |
| ^{a}Inhibition of HSV replication (1 PFU per cell in 5% (v/v) serum) was measured using a viral yield-reduction assay. Activity against HCMV was the ability to prevent formation of microscopic viral plaques. Cell growth inhibition was evaluated using a protein-dye binding assay. Results are mean values and standard error obtained from experiments replicated at least once. NA indicates that no activity was observed. The numbers in parenthesis are | | | | | |
| # the percent inhibition observed at the highest concentration tested. ND indicates that a value was not determined. | | | | | |

The effect of compounds on host cell replication (CC₅₀), was measured using Sulforhodamine B-staining and the spectrophotometric method originally developed for the NCIs's in vitro anti-cancer drug screening program (P. Skehan et al., J. Natl. Cancer. Inst. 82, 1107-1112 (1990)). The CC₅₀ value is the concentration of compound that inhibited actively replicating cells by 50% of control respectively after two days of continuous treatment.

Analysis of HSV proteins. Viral protein synthesis was examined using pulse labeling, SDS-PAGE gel separation and phosphor-imaging. Vero cells were infected and treated under conditions specified below and in FIG. 5. After 17 hours of infection, 10 μCi/mL TRAN³⁵S-LABEL ™ was added directly to culture medium and incubation continued for 30 more minutes. Radio-labeled cell cultures were carefully washed with PBS pre-warmed to 37° C. then lysed at 2,000,000 cells per mL in the same buffer supplemented with 2% (w/v) SDS. After denaturing cell lysates in Laemmli loading buffer, total proteins recovered from 30,000 cells were separated using either 8% (FIG. 5B) or 10% (FIG. 5D) PAGE-SDS gels (U. Laemmli, Nature, 227,680-685 (1970)). Proteins were transferred to nitrocellulose and the synthesized proteins were visualized and quantified using a STORM phosphorimager (Molecular Devices, Sunnyvale CA) and the supplied ImageQuant software according to the manufacturers instructions (FIG. 5, panels B and C). Protein load between samples was compared qualitatively by visual examination of filters stained with India-Ink. A complementary approach for assessing drug effect on viral proteins involved immune detection on a Western-blot using conditions and reagents described previously (Akanitapichat and Bastow, supra 2002). A representative result is shown in FIG. 5D.

Statistical analysis. The program Prism™ version 3 (Graphpad Software, Inc., Sand Diego, Calif.) was used for graphing and statistical analysis of study results.

Anti-HCMV activity of 3,7-dimethoxy-1-hydroxyacridone (2). 3,7-Dimethoxy-1-hydroxyacridone (2), was originally defined as an inactive analog of 1,3,7-trihydroxyacridone, a novel DNA topoisomerase II inhibitor with modest anti-HSV activity (ED₅₀ of 40 μM and 3-fold selectivity; Lowden, supra 1995, J. Vance and K. Bastow, Biochem. Pharmacol. 58, 703-708 (1999); Akanitapichat et al., supra 2000). Subsequent work involving random screening for anti-HCMV agents identified 2 as a candidate inhibitor. The results in FIG. 2A show the activity of 2 measured using a plaque-elimination assay. Compound 2 effectively blocked HMCV plaque formation with an ED₅₀ value of 1.4 μM (0.5 μg/mL). Compound 2 was also examined as an inhibitor of cell replication and was inactive at 50 μM against HEL, Vero and an assortment of human tumor cell lines (Table 1 and data not shown). Therefore compound 2 is a selective (greater than 35-fold) anti-HCMV agent with activity comparable to recently reported values of clinical agents ganciclovir and cidofovir (R. Zhou et al., J. Med. Chem., 40, 802-810 (1997); A. Martinez et al., J. Med. Chem., 43, 3218-3225 (2000)). Subsequent work using higher viral loads showed that compound 2 did not protect against HCMV cytopathogenicity unless HEL cells were pretreated prior to viral infection. A representative result obtained using a prior exposure of 12 days is shown in FIG. 2B. The culture of cells in concentrations of 2 as low as 0.5 μM subsequently afforded some amelioration of HCMV cytopathogenicity. Interestingly, the protective effect at 5 μM was even apparent in the absence of sustained treatment post-infection. The pre-treatment dependence of 2 at higher viral loads is difficult to interpret without understanding mechanism but the significant activity against HCMV replication prompted the exploration of structure-activity through analog synthesis.

Antiviral activities of the 3,5-regioisomer (5) and the parallel series (6-15) Preliminary evaluation of the parallel series as anti-herpes agents showed that several inhibited HCMV replication but unlike 2, four of the active analogs inhibited HSV replication also. Therefore antiviral testing was expanded to include examples of the Citrus alkaloids that exhibit a similar dual anti-herpes activity (N. Yamamoto et al., Antiviral Res., 12, 21-36 (1989)), as well as clinically useful viral DNA polymerase inhibitors with activity against HSV (ACV), or against both HSV and HCMV (PFA). The results obtained for a fixed concentration using a plaque-elimination assay are shown in FIG. 3. Control compounds (ACV, PFA, 1, 1,3,7-trihydroxyacridone, Citrusinine-I and Citpressine-I gave the expected pattern and level of activity based on published work (G. Elion et al., Proc. Nat. Acad. Sci. USA. 74, 5716-5720 (1977); E. Helgestrand et al., Science, 201, 819-821 (1978); Yamamoto et al., supra (1989); Akanitapichat and Bastow, supra (2002)). Compound 2 (10 μM) was designated as a specific HCMV-inhibitor because it was without effect on HSV even at the highest concentration (100 μM) tested (data not shown). Interestingly, the 3,5-dimethoxy regioisomer (5), was inactive against herpes replication at concentrations that did not inhibit host cell replication (mean CC₅₀ of 33 μM), suggesting that the C-7 methoxy substituent was important for the anti-HCMV activity of 2. The two acridone scaffolds used for parallel synthesis were either inactive (compound 3) or only inhibited HSV replication (4). However, compound 4 was not acting as a highly selective antiviral agent because it had significant activity against Vero replication (CC₅₀ of 25 82 M). Four of the C-3 variable-alkoxylated compounds (6-8 and 10), inhibited the replication of both types of herpes virus equally, with the 3-isopropoxy- (8) and the 3-allyloxy-(10) analogues almost completely preventing the formation of microscopic viral plaques. The 3-benzyloxy derivative 9, inhibited HCMV but not HSV-2 replication and an ED₅₀ of 6.9±0.6 μM was subsequently determined. Of the five actives bearing a methoxy substituent at C-7, compound 9 was the least efficacious being about five-fold less active than 2. Significantly, none of the 3-alkoxylated parallel series significantly inhibited host cell replication (CC₅₀ values greater than 50 μM) thereby showing they were acting selectively (at least 8-fold), as viral inhibitors. Of the C-7 variable alkoxylated compounds (11-15), the isopropoxy-derivative (13) was active against HCMV in the screen but a CC₅₀ of 12 μM against HEL replication was subsequently determined. In general, the C-7 variable parallel series comprised of either inactive or weakly active antiviral agents and were not evaluated further. A quantitative comparison of anti-herpes activity between compounds 1, 2 and 10, Citrusinine-I, AVC and PFA was conducted and results are given in Table 1. ACV was four-fold less active against HSV-2 and was inactive against HCMV, consistent with the known activity spectrum of the drug; the activity profile of PFA was also consistent (Elion et al., supra; Helgestrand et al., supra). Citrusinine-I was a selective inhibitor of HSV-2 and HCMV replication but was inactive against the HSV-1 (KOS) strain. Yamamoto et al., reported activity against both HSV sub-types but selectivity was only apparent since cell growth inhibition was tested against a cell line other than the host. The activity spectrum of 5-chloro-1,3-dihydroxyacridone (1), against HSV-1 and HCMV was similar to previous results (Akanitapichat et al., supra 2000) and I inhibited the yield of HSV-2 with an ED₅₀ value of 3.8±0.3 μM in the present study. By way of comparison, 3-allyloxy-1-hydroxy-7-methoxyacridone (10) inhibited replication of HSV-1 and HSV-2 with 41- and 24-fold selectivity respectively and against HCMV, the analog was at least as active as the 3,7-dimethoxy lead (2),

Parameters influencing the anti-HSV activity of C-3 variable alkoxylated analogs. Previous work with 1,3-dihydroxyacridone derivatives showed that activity against HSV is dependent on both multiplicity and serum concentration, the latter variable likely due to serum protein-binding (Akanitapichat et. al. supra 2000). We observed that compounds 6-8 and 10 did not protect against HSV cytopathogenicity under the viral yield assay condition and wondered whether serum concentration and/or viral load could influence the activity of the new compounds also. Results obtained for the 3-allyoxy-derivative 10, are shown in FIG. 4. Compound 10 inhibited the production of cell-associated HSV-1 and this activity was dependent on viral load but only at concentrations of 5 μM and higher (ie the ED₅₀ value was not changed; FIG. 4, panels A and B). In contrast, the concentration of serum in culture medium did not affect the inhibition of cell-associated virus (FIG. 4, compare panel A to panel B). However, both viral load and serum concentration were identified as important variables affecting the amount of virus released into medium in the presence of compound 10 (FIG. 4, compare panel C to panel D). The study results also show that viral release was inhibited more actively than the intracellular viral replication (compare panels A to C and B to D) and under the low viral load and low serum treatment condition (FIG. 4 panel D), the ED₅₀ concentration for inhibition of viral release was actually decreased about six-fold. On the basis of the results, the inhibition of viral release must have contributed to the antiviral efficacy of 10, particularly under the plaque-reduction assay condition. Consistent with this interpretation, compound 8 (at 10 μM in 2% (v/v) serum), inhibited a low multiplicity (0.001 PFU per cell) HSV-2 infection. Treatment completely prevented viral release up to forty-eight hours after infection while the production of cell-associated virus was inhibited by 80% over the same period (data not shown).

Effect of the parallel series on HSV late proteins. The production of HSV proteins are temporally regulated, with the class designated as “late” being dependent upon viral DNA synthesis (R. Honess and Roizman, J. Virol. 14, 8-19 (1974)). Citrusinine-I, PFA and the active metabolite of ACV all interfere with HSV DNA replication (the latter two acting in a direct fashion) and thereby prevent normal production of late viral proteins (Helgestrand et al., supra, Yamamoto et al., supra, P. Furman et al., J. Virol., 32, 72-77 (1979)). By way of comparison and as an approach to delineate a general mode of action, the effects of compounds 6-8 and 10 on HSV proteins were investigated. The results in FIGS. 5 A-C show the inhibition of HSV-2 replication and late protein synthesis with Citrusinine-I as a positive control treatment. Although viral replication was inhibited by about 90% of control by 6-8 and 10 (FIG. 5A), viral protein synthesis was inhibited by only 40-45% (FIG.5 B and C). In contrast, Citrusinine-I inhibited replication and late protein synthesis equally, to about 10% of control values. The activity of compound 10 was also compared to ACV against HSV-1 replication and for effects on viral protein accumulation (Citrusinine-I was not a selective inhibitor of the viral strain used in this work; Table 1). As expected, the level of viral proteins produced from the culture was dependent upon viral load. Compound 10 had no effect on viral protein accumulation under any condition, despite inhibiting viral replication by 64-95% (FIG. 5D). In contrast, when ACV was used as a positive control, the accumulation of HSV-1 proteins was abolished. The results show that compounds 6-8 and 10 at high concentration and in the presence of moderate serum did interfere with normal HSV-2 protein synthesis but this action did not correlate with antiviral activity. However, no effect on viral protein levels was apparent when compound 10 was tested against HSV-1 at a two-fold lower concentration. Overall, the findings suggest that the mechanism of compounds 6-8 and 10 is fundamentally different from either Citrusinine-I or ACV.

The foregoing is illustrative of the present invention, and is not to be construed as limiting thereof. The invention is defined by the following claims, with equivalents of the claims to be included therein.

1. A method of treating a beta-herpes virus infection in a subject in need thereof, comprising administering to said subject a compound of Formula I: 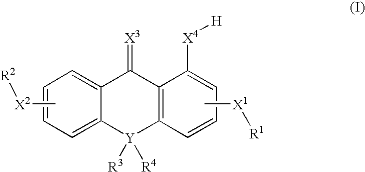
or a pharmaceutically acceptable salt thereof in an amount effective to treat said infection, wherein:
R¹ and R² are each independently selected from the group consisting of H and alkyl;
X¹, X², X³ and X⁴ are each independently selected from the group consisting of O and S;
Y is selected from the group consisting of N, O, S and C;
R³ is selected from the group consisting of H and alkyl, subject to the proviso that R³ is absent when Y is O or S; and
R⁴ is selected from the group consisting of H and alkyl, subject to the proviso that R⁴ is absent when Y is O, S or N.

2. The method of claim 1, wherein said virus is selected from the group consisting of herpes virus 6, herpes virus 7, and human cytomegalovirus.

3. The method of claim 1, wherein R¹ and R² are each H or methyl.

4. The method of claim 1, wherein X¹, X², X³ and X⁴ are O.

5. The method of claim 1, wherein Y is N.

6. The method of claim 1, wherein Y is O.

7. The method of claim 1, wherein Y is S.

8. The method of claim 1, wherein Y is C.

9. The method of claim 1, wherein R³ and R⁴ are H or methyl.

10. A method of treating an alpha-herpes virus infection in a subject in need thereof, comprising administering to said subject a compound of Formula II: 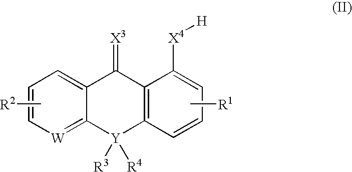
or a pharmaceutically acceptable salt thereof in an amount effective to treat said infection, wherein:
W is selected from the group consisting of N and CR⁵;
R¹, R² and R⁵ are each independently selected from the group consisting of H, alkyl, hydroxy, alkoxy and halo;
X³ and X⁴ are each independently selected from the group consisting of O and S;
Y is selected from the group consisting of N, O, S and C;
R³ is selected from the group consisting of H and alkyl, subject to the proviso that R³ is absent when Y is O or S; and
R⁴ is selected from the group consisting of H and alkyl, subject to the proviso that R⁴ is absent when Y is O, S or N.

11. The method of claim 10, wherein said virus is selected from the group consisting of herpes simplex virus, herpes virus 8, Varicella-Zoster virus and herpes virus simiae.

12. The method of claim 10, wherein W is N.

13. The method of claim 10, wherein W is CR⁵.

14. The method of claim 10, wherein R¹, R² and R⁵ are each independently selected from the group consisting of H and methyl.

15. The method of claim 10, wherein X³ and X⁴ are each O.

16. The method of claim 10, wherein Y is N.

17. The method of claim 10, wherein Y is O.

18. The method of claim 10, wherein Y is S.

19. The method of claim 10, wherein Y is C.

20. The method of claim 10, wherein R³ and R⁴ are each H or methyl.
